(19) United States
(12) Patent Application Publication
(10) Pub. No.: US 2024/0409876 A1
(43) Pub. Date: Dec. 12, 2024

(54) LIQUID COMPOSITION OF GRAM-NEGATIVE BACTERIA

(71) Applicant: Evonik Operations GmbH, Essen (DE)

(72) Inventors: Stefan GILCH, Dülmen (DE); Ina VIERHAUS, Velen (DE); Jennifer WIEGERS, Datteln (DE); Julia NIEWALDA, Selm (DE); Sabrina AOUAG, Essen (DE); Linda MICHEEL, Dorsten (DE); Eva-Maria ECKL, Marl (DE); Jochen KLEINEN, Heinsberg (DE)

(73) Assignee: Evonik Operations GmbH, Essen (DE)

(21) Appl. No.: 18/699,618

(22) PCT Filed: Oct. 5, 2022
(86) PCT No.: PCT/EP2022/077654

§ 371 Date: Apr. 9, 2024

(30) Foreign Application Priority Data
Oct. 14, 2021 (EP) 21202623.1

(51) Int. Cl. C12N 1/04; C12N 1/20; C12R 1/19; C12R 1/39; C12R 1/40

(52) U.S. Cl. CPC C12N 1/04; C12N 1/20; C12R 2001/19; C12R 2001/39; C12R 2001/40

(57) ABSTRACT

The present invention relates to a method of producing a composition of Gram-negative bacteria, the method comprising
(a) subjecting a wet mass of Gram-negative bacteria to spray drying; and
(b) contacting the spray-dried Gram-negative bacteria from step (a) to a compound,
wherein the compound has a general formula (I) of,

R¹O—[(C₂H₃R²)—O]ₙ—H Formula (I)
where
R¹ is a monovalent aliphatic radical having 1 to 22, preferably 2 to 10, especially 3 to 4, carbon atoms;
R² is in each case independently a hydrogen radical or a methyl radical; and
n is a number from 1 to 300, preferably from 5 to 100, especially from 10 to 30,
with the proviso that at least one R² radical is a methyl radical.

### FIELD OF THE INVENTION

The present invention relates a method of producing a liquid composition of Gram-negative bacteria, a method of improving storage stability of Gram-negative bacteria and a liquid composition of Gram-negative bacteria. In particular, the Gram-negative bacteria is first subjected to spray drying before contacting the spray-dried Gram-negative bacteria to a polyether.

### BACKGROUND OF THE INVENTION

Gram-negative bacteria are known to have a high metabolic diversity and can thus be used in a variety of fields such as agriculture, personal care, health care and animal health. For example, the use of beneficial Gram-negative bacteria as alternatives to chemical pesticides and synthetic fertilizers in agricultural production is an area of increasing interest. Inoculant compositions comprising plant promoting bacteria or nitrogen fixing bacteria are well known and a commonly used biofertilizer. Rhizobacteria, a Gram-negative bacterium, are commonly applied as inoculants and include nitrogen-fixers and phosphate-solubilizers which enhance the availability of the macronutrients nitrogen and phosphorus to the host plant. The most commonly applied rhizobacteria are Rhizobium, Bradyrhizobium and closely related genera. Rhizobium and Bradyrhizobium are nitrogen-fixing bacteria that form symbiotic associations within nodules on the roots of legumes. Such behaviour increases host nitrogen nutrition and is important to the cultivation of soybeans, chickpeas and many other leguminous crops. Rhizobium also reduces the need of anthropogenic N-fertilization and thus lowers indirect CO₂ emissions caused by the Haber-Bosch-Process.

However, since Gram-negative bacteria (in contrast to Gram-positive bacteria and/or fungi) cannot form persistence forms, so-called spores, and also only have a thin, single layer murein envelope to stabilize the cell membrane, the stability of Gram-negative bacteria is limited. In most of the areas of application however, a high level of stability both in storage and after use is required for customers to be convinced to use the Gram-negative bacteria and also to improve economic efficiency. In particular, bacterial inoculants are, only effective when, after application, the microorganisms are readily able to survive and thrive in soil conditions. Application of bacterial inoculant formulations and the soil environment itself subjects these inoculants to a variety of stresses, including temperature, mechanical, light, oxidative and osmotic stress, all of which impact the survivability of the bioactive. Thus, a further limitation to the use of bacterial inoculants is a low organism survival rate.

Many available products in the field of agricultural use have a shelf life based on their short-lived stability of only a few days to a few weeks maximum and have to be stored at low temperatures throughout this short period. Also, the products currently sold on the market are usually available as aqueous solutions or supported on peat or clay. All these products have a high-water activity (aW) that keeps the Gram-negative organisms metabolically active throughout their storage. Therefore, to prevent the bacteria from dying and to ensure high stability/durability of the Gram-negative bacteria during their storage period, an appropriate carbon source (usually sugar and/or biological extracts) is currently being added or the product containing the metabolically active Gram-negative bacteria must be stored at low temperatures in order to slow down the metabolic processes which makes the use of these Gram-negative bacteria costly and inconvenient.

Further, both chemical and biological active ingredients (pesticides, plant strengtheners, fertilizers) are often applied to the target site together with effect enhancers (additives, adjuvants). Dispersing additives, emulsifiers, defoamers etc. improve the physical properties of the product or concentrate. Spreading agents and adhesives ensure an improved effect or effectiveness of the chemical or biological active substances. However, since many effect enhancers only have a low level of biocompatibility, they can only be added in low concentrations (0.1 to 2%) as a tank mix. As a result, due to a lack of biocompatibility, it is often not possible to use the optimal concentration (about 40% or more adjuvant) of the enhancer in interaction with Gram-negative organisms in a single can. The effect enhancer and the Gram-negative cells are first brought into contact with one another in the tank. Furthermore, errors can be made by the user when the active substance and the effect enhancer are mixed manually in the tank, which lead to a partial reduction or a complete lack of effectiveness. Farmers also prefer in-can formulations because they only have to add one component (e.g. water), when the bacteria and adjuvant are already provided in the single can compared to individual additions of bacteria, adjuvant and water.

Accordingly, there remains a need in the art for a formulation of Gram-negative bacteria for storage that maintains the stability of the bacteria without jeopardizing on the efficiency of using the bacteria in the different fields. In particular, there is a need in the art for a formulation of Gram-negative bacteria with a durability/stability of at least 1-2 years at ambient temperature and increased humidity and a method for making these formulations of Gram-negative bacteria that provide improved bacterial yield and storage survivability.

### FIGURES OF THE DESCRIPTION

FIG. 1 are graphs showing the stabilization of Pseudomonas fluorescens using the method according to any aspect of the present invention. FIG. 1(A) are the survival rate and storage results of direct liquid formulation and FIGS. 1(B) and (C) are the survival rate and storage results of spray dried Pseudomonas fluorescens then formulated in liquid 1:10 adjuvants.

FIG. 2 are graphs showing the stabilization of Azospirillum brasilense using the method according to any aspect of the present invention. FIGS. 1(A) and B are the survival rate and storage results of direct liquid formulation and FIG. 1(C) is the survival rate and storage results of spray dried Azospirillum brasilense then formulated in liquid 1:10 adjuvants.

FIG. 3 shows the stabilization of Azotobacter chroococcum using the direct liquid formulation.

FIG. 4 are graphs showing the stabilization of Bradyrhizobium japonicum using the method according to any aspect of the present invention. FIG. 1(A) is the survival rate and storage results of direct liquid formulation and FIG. 1(B) is the survival rate and storage results of spray dried Bradyrhizobium japonicum then formulated in liquid 1:10 adjuvants.

FIG. 5 are graphs showing the stabilization of Escherichia coli using the method according to any aspect of the present invention. FIG. 1(A) is the survival rate and storage results of direct liquid formulation and FIG. 1(B) is the survival rate and storage results of spray dried Escherichia coli then formulated in liquid 1:10 adjuvants.

FIG. 6 are graphs showing the stabilization of Pseudomonas putida using the method according to any aspect of the present invention. FIG. 1(A) is the survival rate and storage results of direct liquid formulation and FIG. 1(B) is the survival rate and storage results of spray dried Pseudomonas putida then formulated in liquid 1:10 adjuvants.

FIG. 7 is a graph showing the stability of the adjuvants in combination with Pseudomonas fluorescens when the cells are pretreated differentially (spray dried or not).

### DESCRIPTION OF THE INVENTION

The present invention attempts to solve the problems above by providing an improved means of storage and transport of Gram-negative bacteria. In particular, any aspect of the present invention may be used to provide a composition of spray-dried Gram-negative bacteria as the main active ingredient mixed with at least one carrier which leads to improved handling and shelf life of the Gram-negative bacteria compared to the prior art. In particular, an increased biological effectiveness of the Gram-negative bacteria active ingredient and/or bioavailability over a longer period may be achieved in comparison with the prior art. It has surprisingly been found that producing a composition of Gram-negative bacteria using a method of first spray-drying the Gram-negative bacteria and then combining the dried cells with a polyether or compound of Formula (I) as a carrier for the cells may solve the problems above. In particular, the Gram-negative bacteria, the active ingredient and the polyether or compound of Formula (I), the activity enhancer, can be made up as a concentrate in a common premix and only have to be added to the tank in a single dose. Thus, allowing the use of an optimal concentration of the enhancer in interaction with the Gram-negative bacteria and also reducing chances of errors made by the user who usually mixes the Gram-negative bacteria with the activity enhancer in the tank manually as this step of manual mixing is skipped.

Further, the polyether or compound of Formula (I) has a low water activity and thus growth of not only the Gram-negative bacteria is stopped but also that of unwanted growth of contaminating microorganisms, such as fungus and mould are prevented. This also helps increase the shelf-life of the Gram-negative cells.

According to one aspect of the present invention, there is provided a method of producing a liquid composition of Gram-negative bacteria, the method comprising
(a) subjecting a wet mass of Gram-negative bacteria to spray drying; and
(b) contacting the spray-dried Gram-negative bacteria from step (a) to a compound, to form the liquid composition of Gram-negative bacteria,
wherein the compound has a general formula (I) of,

R¹O—[(C₂H₃R²)—O]ₙ—H Formula (I)
where
R¹ is a monovalent aliphatic radical having 1 to 22, preferably 2 to 10, especially 3 to 4, carbon atoms;
R² is in each case independently a hydrogen radical or a methyl radical; and
n is a number from 1 to 300, preferably from 5 to 100, especially from 10 to 30,
with the proviso that at least one R² radical is a methyl radical.

The composition of Gram-negative bacteria may be a liquid formulation that protects the cells from penetrating water for a long time, which would lead to a decrease in stability. Spray-drying the Gram-negative bacteria and then combining the spray-dried Gram-negative bacteria with the compound according to any aspect of the present invention leads to the desired increase in stability of the bacteria. Also, spray drying is able to reduce the aW in the cells from 1 to about 0.2-0.3 very quickly compared to other methods known in the art. Due to the biocompatibility of the mixture of hydrophobic, partially water-insoluble polyglycerol ester and emulsifier, the ratios of all components present are freely adjustable. The viscosity of the liquid composition of the Gram-negative bacteria, the final product also remains low in all mixing ratios. That enables the final product to be used up without any final product being wasted in the tank or spray containers, draining from the tanks and spray containers is also quick. Also, mixing is easy to handle and takes only little time. Further, no further emulsifiers have to be added outside of what is in the original mixture as, the mixture according to any aspect of the present invention is water-soluble. Addition of other emulsifiers and/or any other (bio-)active components may also have a toxic effect on the Gram-negative bacterial cells. Therefore, it is advantageous when such additions can be avoided.

The liquid composition of Gram-negative bacteria having a low aW offer the added benefit of improved stability and may eliminate the need for refrigeration due to dormant state of the cells. Such formulations also provide a lower risk of contamination.

Step (a) of spray-drying the Gram-negative bacteria, may be carried out in the presence of at least one additive. In particular, the Gram-negative bacterial cells may be stabilized during the dehydration process by introduction of at least one additive to the wet mass of the Gram-negative bacterial cells prior to subjecting the cells to spray-drying. The additive introduced to the wet mass of cells may be selected from at least one (i) silica, (ii) sugar with low water activity (aW value) and/or (iii) adhesive with low water activity (aW value). Any type of silica may be used according to any aspect of the present invention. In particular, silica may be used as a defined hydrophilic carrier with water-absorbing properties (maintenance of low water activity; aW). In one example, the silica used may be selected from the group consisting of Sipernat® 50, Sipernat® 50S, Sipernat® 22, Sipernat® 22S, Sipernat® 880, Zeolex® 7, Spherilex® 148, Zeolex® 23, and Zeolex® 23a. In particular, the silca used according to any aspect of the present invention may be Sipernat® 50, a carrier silica with high absorbency.

The (ii) sugar may be selected from the group consisting of isomalt, sucrose, isomaltulose, maltitol, erythritol and mannitol. In particular, the (ii) sugar may be isomalt. In one example, the (ii) sugar may be Risumalt® an isomalt. The (iii) adhesive with maintenance of low water activity used as a glass former may be selected from the group consisting of Gummi Arabicum, Xanthan and mica powder. In particular, the adhesive may be Gummi Arabicum. More in particular, the (ii) sugar may be Risumalt® and the (iii) adhesive may be Gummi Arabicum.

This step of spray drying is an essential part of the process, as rapid dehydration together with the vitrification of sugar (for example Risumalt®) is necessary for a high survival rate, stability, and shelf life of the Gram-negative bacteria.

The spray-drying may also be carried out in the presence of the medium in which in the Gram-negative cells were cultured. In particular, the medium may be Lysogeny broth (LB). More in particular, LB may be LB-Luria with 0.5 g/L NaCl. A skilled person would easily be able to prepare the LB medium used according to any aspect of the present invention.

In one example, the spray-drying according to any aspect of the present invention is carried out in the presence of a silica, a sugar and/or an adhesive. In particular, the spray-drying is carried out in the presence of a silica and a sugar. More in particular, the spray-drying is carried out in the presence of a silica and an isomalt. Even more in particular, the spray-drying is carried out in the presence of a silica, an isomalt and an adhesive. In particular, the spray-drying is carried out in the presence of a silica, an isomalt and Gummi Arabicum.

The spray-drying according to any aspect of the present invention is carried out in the presence of a silica, a sugar, an adhesive and/or LB. In particular, the spray-drying is carried out in the presence of a silica, a sugar, an adhesive and LB. More in particular, the spray-drying is carried out in the presence of a silica, an isomalt, an adhesive and LB. Even more in particular, the spray-drying is carried out in the presence of a silica, an isomalt, Gummi Arabicum and LB.

The spray-drying according to any aspect of the present invention may be carried out using any spray-dryer. In one example, a spray-dryer, Mini Spray Dryer B-290 (Büchi Labortechnik AG, Switzerland) may be used. In particular, the wet mass of Gram-negative bacteria may be spray-dried by a spray-dryer, whereby the spray-dried Gram-negative bacteria according to any aspect of the present invention may be obtained.

In step (a) according to any aspect of the present invention, the spray dryer may first be preheated with a very low fan speed. Spray drying may be achieved by allowing the inlet air temperature to be not so high so that the Gram-negative cells will survive the temperature. In particular, the inlet air temperature may be less than or equal to about 80° C., less than or equal to about 50° C., at about 30°-50° C. The spray drying may be with gas flow. The outlet air temperature may be less than or equal to about 55° C., less than or equal to about 54, or 50° C., at about 30°-55° C. The drying time may be considered to be proportional to the surface area of the silica and the control of water activity is inversely proportional to surface area of the silica. In one example, the spray-drying process of step (a) may be carried out using Büchi B-290 spray dryer with 10% pump power.

The composition of Gram-negative bacteria obtained after step (a) of spray drying has a water activity (aW) of about 0.01 to 0.5. The activity of water (aW) is a thermodynamic parameter. It is a measure of the amount of water available for chemical, biochemical and microbial reactions of samples, such as aqueous solutions and food, and can also be used to characterize the carrier (mixture) compositions. The water activity is given as a value and is defined as the ratio of the water vapour pressure above the sample (p) to the water vapour pressure of pure water (po) at the same temperature, a=p/po. The water activity corresponds to 1/100 of the relative equilibrium humidity (RGF). Equilibrium relative humidity is also referred to as equilibrium relative humidity (ERH). Pure water has an a_{w} of 1, and any addition of water-binding substances results in lowering of the a_{w} below 1. It is preferable that the a_{w} of the carrier composition is less than 0.4, particularly less than 0.3, more particularly less than 0.25. Water or aqueous solutions are generally unsuitable for use as carrier for active microbiological ingredients owing to their high water activity. Methods of determining the a_{w} value are known to the person skilled in the art. The a_{w} value is preferably determined as described below.

To determine the activity of water of a sample, the air humidity is measured directly above a sample after attainment of equilibrium relative humidity (partial water vapour pressure differential). Equilibrium relative humidity (ERH) is measured in % relative humidity and is related to the a_{w} value by the following relationship: a_{w}=ERH/100. The activity of water in the compositions was determined using the LabMaster-aW neo from Novasina.

In particular, the spray-dried Gram-negative bacteria obtained after step (a) of spray drying has a water activity (aW) of 0.01 to 0.45, 0.01 to 0.4, 0.01 to 0.35, 0.01 to 0.3, 0.01 to 0.25, 0.01 to 0.2, 0.05 to 0.5, 0.05 to 0.45, 0.05 to 0.4, 0.05 to 0.35, 0.05 to 0.3, 0.05 to 0.25, 0.05 to 0.2, 0.05 to 0.15, 0.05 to 0.1, 0.1 to 0.5, 0.1 to 0.45, 0.1 to 0.4, 0.1 to 0.35, 0.1 to 0.3, 0.1 to 0.25, 0.1 to 0.2, 0.1 to 0.15, 0.15 to 0.5, 0.15 to 0.45, 0.15 to 0.4, 0.15 to 0.3, 0.15 to 0.25, or 0.15 to 0.2. More in particular, the spray-dried Gram-negative bacteria obtained after step (a) of spray drying has a water activity (aW) of about 0.01, 0.05, 0.1, 0.15, 0.2, 0.25, 0.3, 0.35, or 0.4.

The spray-dried Gram-negative bacterial cells of step (a) may then be contacted with at least one compound to form the liquid composition of Gram-negative bacteria according to any aspect of the present invention. The compound may be considered an anhydrous additive that may be used as a carrier for the Gram-negative bacteria and leads to an increased shelf life/storage stability of the cells compared to water-dispersible powders or granules. In particular, the compound of the formula (I) as carrier for the Gram-negative bacteria leads to improved storage stability of the Gram-negative bacteria. The compound of the formula (I) additionally shows sufficiently low viscosity even at low temperature. This leads to better disability, simpler handling and faster and equal mixing of the Gram-negative bacteria or of the final product, the liquid composition of Gram-negative bacteria. The compound of the formula (I) is additionally self-emulsifiable or water-soluble and hence has good dilutability with water. It is therefore unnecessary to use further additives that improve the solubility or dispersibility of the Gram-negative bacteria in the spray liquor. In particular, the compound of Formula (I) is able to not only conserve colony-forming unit (CFU) over time, but also increases the CFU-number (compared to aqueous solutions) by disrupting agglomerations of the Gram-negative bacteria. More in particular, the compound of Formula (I) is able to bring about the disruption of agglomeration of the bacteria, where the compounds are able to dissolve/disperse such agglomerations without being a surfactant itself. As a consequence, the user needs one component less.

Correspondingly, the liquid composition of Gram-negative bacteria according to any aspect of the present invention also shows improved storage stability, has better dosability and easier handling and also has good solubility or good dispersibility in water.

The composition of Gram-negative bacteria according to any aspect of the present invention, a liquid formulation, may be diluted for example with water to form a liquid Gram-negative bacteria spray before being used in agriculture (i.e. sprayed on crops). The compound used in step (b) as carrier according to any aspect of the present invention can be diluted for spray applications with other additives (e.g. SP133, S301, etc.) in any ratio to adjust/modulate the optimal additive: bacteria (CFU) concentration. This is within the common general knowledge of a skilled person. Uncoupling of bacteria/additive concentrations might also be interesting for other applications than agriculture. It is within the common general knowledge of a skilled person to vary the ratio of bacteria to additive to arrive a final product that is suitable for other applications in healthcare, the food industry, household care, personal care, paintings, animal welfare and the like. Any additive known in the art may be used in combination with the liquid composition of Gram-negative bacteria according to any aspect of the present invention. In particular, the additive may be selected from the group consisting of Break-Thru® S 200, Break-Thru® S 233, Break-Thru® S 240, Break-Thru® S 279, Break-Thru® S 301, which are all trisiloxanes, Break-Thru® OE 441, Break-Thru® OE 446, which are all polyether siloxanes, Break-Thru® SP 133, Break-Thru® Vibrant, Surfynol® 104, Surfynol® 420, Surfynol® 485, which are all organic surfactants, Break-Thru® DA 646, Break-Thru® DA 647, Break-Thru® DA 655, which are polyethers, Break-Thru® EM O 5, Break-Thru® EM O 7, Break-Thru® EM V 20, which are ethoxylated organic surfactants, Tego® STO 85 V, an ethoxylated sorbitan oleate, Tego® SML, a sorbitan laurate, Tego® SML 20, an ethoxylated sorbitan laurate, Polysorbat® 40, a ethoxylated sorbitan palmitate, Polysorbat® 80 and Tego®SMO 80 V, which are ethoxylated sorbitan oleates, Tego® STO and Tego® SMO, which are sorbitan oleates, Tego® SMS, a sorbitan stearate, Rewomid® DC 212, a fatty acid diethanol amine, Rewocare® CH 40, a polyoxyethylene-40 hydrogenated castor oil, Tegotens® EC 11, an end-capped fatty alcohol ethoxylate, Tomadol® 91-6, Tomadol® 1-3, Tomadol® 1-5, Tomadol® 1-7, Tomadol® 1-9, Tomadol® 23-1, Tomadol® 25-9, which are all fatty alcohol ethoxylates, Varonic® 14, a polyglycerol ester, Abil® Care 85 MB, a modified siloxane, Tego® Solve 51 MB, a polyglycerol ester, PEG 6000 Distearate, a polyethylene glycol distearate, PEG 6000, PEG 400, PEG 200, polyethylene glycols, water, nutrients, Silwet® L-77, a trisiloxane, Pluronic® PE 6400, a polyether, Marlipal® 013/30, Marlipal® 013/90 and Marlipal® 013/120, fatty alcohol ethoxylates, and Atlox® 4916, a polymeric emulsifier. In particular, the nutrients may be selected from sugar, soy peptone, milk powder, glycerol and the like.

In addition, the carrier can be produced sustainably from renewable raw materials and is also largely biodegradable. The carrier therefore shows a particularly good profile of properties.

The term ‘carrier’ refers to the compound of Formula I used according to any aspect of the present invention. The carrier is able to transport the Gram-negative bacterial cells according to any aspect of the present invention to its final target (for example in agriculture to a part of the plant/crop). The carrier used according to any aspect of the present invention has also been disclosed in PCT/EP2021/060659. The Gram-negative bacteria is thus dissolved or dispersed in the carrier. The carrier additionally assists the dissolving or dispersing of the spray-dried Gram-negative bacteria in an aqueous composition, for example the spray liquor.

The subjects according to the invention will be described hereinbelow without the invention being limited to these exemplary embodiments. If ranges, general formulae or classes of compounds are mentioned hereinbelow, they are intended to comprise not only the corresponding ranges or groups of compounds which are mentioned explicitly, but also all part-ranges and part-groups of compounds which can be obtained by selecting individual values (ranges) or compounds. When documents are cited for the purposes of the present description, the entire content of these is intended to be part of the disclosure of the present invention. When % data are provided hereinbelow, they are, unless otherwise specified, data in % by weight. In the case of compositions, the % data, unless otherwise specified, refer to the total composition. When averages are mentioned, they are, unless otherwise specified, mass averages (weight averages). When measured values are stated hereinafter, then these measured values, unless otherwise specified, were determined at a pressure of 101325 Pa and a temperature of 25° C.

The statement of a mass ratio of, for example, component (a) to component (b) of 0.1 means that a mixture comprising these two components includes 10% by weight of component (a) based on the total of the masses of components (a) and (b).

The compound of the formula (I) has a terminal monovalent aliphatic radical R¹ having 1 to 22, preferably 2 to 10, especially 3 to 4, carbon atoms. By comparison with polyethers that are exclusively OH-terminated, this leads to a distinct improvement in storage stability. R¹ may, for example, be linear or branched, cyclic or acyclic, and saturated or unsaturated. It is preferable that the R¹ radical is an alkyl radical, preferably a radical selected from the group consisting of methyl, ethyl, n-propyl, iso-propyl, n-butyl, iso-butyl, sec-butyl, tert-butyl, n-pentyl (amyl), 2-pentyl (sec-pentyl), 3-pentyl; 2-methylbutyl, 3-methylbutyl (iso-pentyl or iso-amyl), 3-methylbut-2-yl, 2-methylbut-2-yl, 2,2-dimethylpropyl (neopentyl), hexyl, octyl, decyl, dodecyl, myristyl, stearyl, 2-ethylhexyl, 2-propylheptyl, 3,5,5-trimethylhexyl, isononyl, isotridecyl, especially an n-butyl radical.

The compound of the formula (I) has one or more divalent —[(C₂H₃R²)—O]— groups where each R² radical is independently a hydrogen radical or a methyl radical. The divalent —[(C₂H₃R²)—O]— groups are thus alkyleneoxy groups. If R² is a hydrogen radical, i.e. in the case that: R²=H, the —[(C₂H₃R²)—O]— group is a —[(C₂H₄)—O]— group, i.e. a —(CH₂—CH₂—O)— group, i.e. an ethyleneoxy group. If, by contrast, R² is a methyl radical, i.e. in the case that: R²=CH₃, the —[(C₂H₃R²)—O]— group is a —[(C₂H₃(CH₃))—O]— group, i.e. a propyleneoxy group. Each propyleneoxy group may independently be in the spatial orientations —(CH₂—CH(CH₃)—O)— or —(CH(CH₃)—CH₂—O)—, but preferably in the spatial orientation —(CH₂—CH(CH₃)—O)—, in the compound of the formula (I), where the compound of the formula (I) should be based on the spatial orientation chosen in formula (I), i.e. a spatial orientation in which the R¹O group is present bonded at the left-hand end and the OH group at the right-hand end of the compound of the formula (I).

The index n is a number from 1 to 300, preferably from 5 to 100, especially from 10 to 30. The index n is thus, for example, a number from 1 to 300, from 2 to 250, from 3 to 200, from 4 to 150, from 5 to 100, from 6 to 81, from 7 to 50, from 8 to 40 and/or from 10 to 30, preference being given in each case to a narrower numerical range over a broader numerical range. When n>300, the viscosity of the compound of the formula (I) is significantly increased and it is therefore not very suitable as carrier since the elevated viscosity worsens the dosability and handling of the active ingredient composition.

In particular, in the compound of the formula (I), 10% to 100%, more in particular 20% to 80%, even more in particular 40% to 60%, of the R² radicals are methyl radicals, the maximum value being 100%. The proportion of propyleneoxy groups based on the total amount of alkyleneoxy groups, i.e. the total amount of propyleneoxy and ethyleneoxy groups together, is thus from 10% to 100%, particularly from 20% to 80%, more particularly from 40% to 60%, the maximum value being 100%. The number of propyleneoxy groups divided by the total number of alkyleneoxy groups, i.e. the total number of propyleneoxy and ethyleneoxy groups together, is thus from 10% to 100%, particularly from 20% to 80%, more particularly from 40% to 60%, the maximum value being 100%.

This is because it has been found that polyethers containing solely ethyleneoxy units and no propyleneoxy units show a high viscosity or are solid, especially at low temperatures, whereas polyethers having solely propyleneoxy units and no ethyleneoxy units or else containing both ethyleneoxy units and propyleneoxy units have a low viscosity across the board.

In one example, a compound of the formula (I)

R¹O—[(C₂H₃R²)—O]ₙ—H Formula (I)

where
R¹ is a monovalent aliphatic radical having 2 to 10 carbon atoms, especially an alkyl radical having 2 to 10 carbon atoms;
R² is in each case independently a hydrogen radical or a methyl radical;
n is a number from 2 to 250;
with the proviso that 20% to 80% of the R² radicals are methyl radicals,
is used

In another example, a compound of the formula (I)

R¹O—[(C₂H₃R²)—O]ₙ—H Formula (I)

where
R¹ is a monovalent aliphatic radical having 2 to 10 carbon atoms, especially an alkyl radical having 2 to 10 carbon atoms;
R² is in each case independently a hydrogen radical or a methyl radical;
n is a number from 5 to 100;
with the proviso that 20% to 80% of the R² radicals are methyl radicals,
is used.

In yet another example, a compound of the formula (I)

R¹O—[(C₂H₃R²)—O]ₙ—H Formula (I)

where
R¹ is a monovalent aliphatic radical having 3 to 4 carbon atoms, especially an alkyl radical having 3 to 4 carbon atoms;
R² is in each case independently a hydrogen radical or a methyl radical;
n is a number from 6 to 81;
with the proviso that 20% to 80% of the R² radicals are methyl radicals, is used.

In a further example, a compound of the formula (I)

R¹O—[(C₂H₃R²)—O]ₙ—H Formula (I)

where
R¹ is a monovalent aliphatic radical having 3 to 4 carbon atoms, especially an alkyl radical having 3 to 4 carbon atoms;
R² is in each case independently a hydrogen radical or a methyl radical;
n is a number from 8 to 40;
with the proviso that 20% to 80% of the R² radicals are methyl radicals,
is used.

In a still further example, a compound of the formula (I)

R¹O—[(C₂H₃R²)—O]ₙ—H Formula (I)

where
R¹ is a monovalent aliphatic radical having 3 to 4 carbon atoms, especially an alkyl radical having 3 to 4 carbon atoms;
R² is in each case independently a hydrogen radical or a methyl radical;
n is a number from 10 to 30;
   with the proviso that 20% to 80% of the R² radicals are methyl radicals,
is used.

In one example, a compound of the formula (I)

R¹O—[(C₂H₃R²)—O]ₙ—H Formula (I)

where
R¹ is an n-butyl;
R² is in each case independently a hydrogen radical or a methyl radical;
n is a number from 2 to 250;
   with the proviso that 20% to 80% of the R² radicals are methyl radicals,
is used.

In yet another example, a compound of the formula (I)

R¹O—[(C₂H₃R²)—O]ₙ—H Formula (I)

where
R¹ is an n-butyl;
R² is in each case independently a hydrogen radical or a methyl radical;
n is a number from 5 to 100;
with the proviso that 20% to 80% of the R² radicals are methyl radicals,
is used.

In one example, a compound of the formula (I)

R¹O—[(C₂H₃R²)—O]ₙ—H Formula (I)

where
R¹ is an n-butyl;
R² is in each case independently a hydrogen radical or a methyl radical;
n is a number from 6 to 81;
with the proviso that 20% to 80% of the R² radicals are methyl radicals,
is used.

In another example, a compound of the formula (I)

R¹O—[(C₂H₃R²)—O]ₙ—H Formula (I)

where
R¹ is an n-butyl;
R² is in each case independently a hydrogen radical or a methyl radical;
n is a number from 8 to 40;
with the proviso that 20% to 80% of the R² radicals are methyl radicals,
is used.

In yet another example, a compound of the formula (I)

R¹O—[(C₂H₃R²)—O]ₙ—H Formula (I)

where
R¹ is an n-butyl;
R² is in each case independently a hydrogen radical or a methyl radical;
n is a number from 10 to 30;
with the proviso that 20% to 80% of the R² radicals are methyl radicals,
is used.

In a further example, a compound of the formula (I)

R¹O—[(C₂H₃R²)—O]ₙ—H Formula (I)

where
R¹ is an n-butyl;
R² is in each case independently a hydrogen radical or a methyl radical;
n is a number from 2 to 250;
with the proviso that 40% to 60% of the R² radicals are methyl radicals,
is used.

In one example, a compound of the formula (I)

R¹O—[(C₂H₃R²)—O]ₙ—H Formula (I)

where
R¹ is an n-butyl;
R² is in each case independently a hydrogen radical or a methyl radical;
n is a number from 5 to 100;
with the proviso that 40% to 60% of the R² radicals are methyl radicals,
is used.

In a still further example, a compound of the formula (I)

R¹O—[(C₂H₃R²)—O]ₙ—H Formula (I)

where
R¹ is an n-butyl;
R² is in each case independently a hydrogen radical or a methyl radical;
n is a number from 6 to 81;
with the proviso that 40% to 60% of the R² radicals are methyl radicals,
is used.

In a still further example, a compound of the formula (I)

R¹O—[(C₂H₃R²)—O]ₙ—H Formula (I)

where
R¹ is an n-butyl;
R² is in each case independently a hydrogen radical or a methyl radical;
n is a number from 8 to 40;
with the proviso that 40% to 60% of the R² radicals are methyl radicals,
is used.

In yet another example, a compound of the formula (I)

R¹O—[(C₂H₃R²)—O]ₙ—H Formula (I)

where
R¹ is an n-butyl;
R² is in each case independently a hydrogen radical or a methyl radical;
n is a number from 10 to 30;
with the proviso that 40% to 60% of the R² radicals are methyl radicals,
is used.

Some further examples of compounds of Formula (I) may at least be found in PCT/EP2021/060659 in Table 2. Each of the examples 10-18 which fall within the compound of Formula (I) may be used according to any aspect of the present invention.

Carrier compositions containing or consisting (essentially) of compounds of the formula (I) according to at least one of these examples are notable for a particularly good profile of properties. They lead to improved storage stability of the active ingredient, have low viscosity even at low temperature and hence enable better dosability and handling of the active ingredient composition, and are additionally self-emulsifiable or water-soluble and hence have good dilutability with water. These carrier compositions are also able to bring about the disruption of agglomerations of the bacteria, where the compounds are able to dissolve/disperse such agglomerations without being a surfactant itself.

In particular, the number-average molar mass of the at least one compound of the formula (I) is at least 300 g/mol, more particularly at least 400 g/mol, even more particularly at least 800 g/mol, especially at least 1200 g/mol. The number-average molar mass of the at least one compound of the formula (I) is from 300 g/mol to 4500 g/mol, particularly from 400 g/mol to 3000 g/mol, more particularly from 800 g/mol to 2000 g/mol, even more particularly from 1200 g/mol to 1500 g/mol. A number-average molar mass within the above-specified ranges leads to an optimal viscosity at low temperatures (e.g. 0° C.) at which biological plant protection products in particular are frequently stored, and also at room temperature (e.g. 25° C.), at which chemical plant protection products in particular are stored. If the viscosity is too high, the active ingredient composition is difficult to dose and handle; if the viscosity is too low, there can be unwanted separation of dispersion phase and dispersed phase, for example settling/sinking of the Gram-negative bacteria.

The polarity, molecular weight and hydrophobicity/hydrophilicity of compounds of the formula (I) can be adjusted such that they are self-emulsifying in water or water-soluble and hence have good dilutability with water. In particular, the HLB value of the at least one compound of the formula (I) is from 0.0 to 14.0, particularly from 3.0 to 10.0, more particularly from 7.0 to 9.5. “HLB” stands for hydrophilic-lipophilic balance. The HLB value can be determined by various prior art methods and is a recognized measure of hydrophobicity/hydrophilicity. The HLB value is determined by the Griffin method (W. C. Griffin: Classification of surface active agents by HLB, J. Soc. Cosmet. Chem. 1, 1949, p. 311-326). The HLB value is calculated here by the formula

$$HLB = 20 \cdot ( 1 - \frac{{m}_{1}}{m} )$$

where mₗ is the molar mass of the lipophilic component of a molecule and m is the molar mass of the entire molecule. The molar mass mₕ of the hydrophilic component of a molecule is correspondingly found using mₕ=m−mₗ. The molar masses are determined by prior art methods; they are particularly determined by mass spectrometry; the lipophilic component or the hydrophilic component is likewise particularly determined from the mass spectrometry results using the stoichiometric principles known to the person skilled in the art. The molar masses can also be calculated from the molecular structure. In the case of compounds of the formula (I), mass of the hydrophilic component is calculated from the total mass of all —[(C₂H₃R²)—O]— groups with R²=H, i.e. from the total mass of all ethyleneoxy groups (oxyethylene groups) present.

It has also been found that, surprisingly, the viscosity and dilutability (self-emulsifiability or water solubility) of the carrier according to any aspect of the present invention can be controlled by mixing of different polyethers.

It is therefore advantageous that at least one compound (A) and at least one different compound (B) are used, where both the at least one compound (A) and the at least one compound (B) are compounds of the formula (I), and where the HLB value of the at least one compound (A) is from 0.0 to 10.0, particularly from 0.0 to 3.0, more particularly from 0.0 to 3.0, and the HLB value of the at least one compound (B) is from 2.0 to 15.0, particularly from 4.0 to 14.0, more particularly from 8.0 to 13.0.

The combination of a compound (A) with HLB=0 that has solely oxypropylene units and no oxyethylene units and a compound (B) with HLB>0 that has both oxyethylene units and oxypropylene units shows particularly advantageous properties as carrier.

In particular, the carrier contains predominantly the at least one compound of the formula (I). It is therefore preferable that the proportion by mass of the at least one compound of the formula (I) is at least 90%, particularly at least 95%, more particularly at least 99%, based on the total mass of the carrier, the maximum value being 100%. It is thus preferable that the proportion by mass of all compounds of the formula (I) is at least 90%, particularly at least 95%, more particularly at least 99%, based on the total mass of the carrier, the maximum value being 100%.

It is particularly advantageous when the composition used as carrier consists (essentially) of the at least one compound of the formula (I), i.e. when the proportion by mass of the at least one compound of the formula (I) is 100%, i.e. corresponds to the maximum value. It is thus preferable that the proportion by mass of all compounds of the formula (I) is 100%, i.e. corresponds to the maximum value.

Processes for preparing compounds of the formula (I) are known to the person skilled in the art. The compounds of the formula (I) are particularly obtained by reacting hydroxy-functional compounds of the formula R¹—OH where R¹ is as defined in formula (I) with propylene oxide (PO) and optionally additionally ethylene oxide (EO). This reaction is an alkoxylation reaction of R¹—OH with PO and optionally additionally EO. The hydroxy-functional compounds of the formula R¹—OH used are aliphatic monofunctional alcohols having 1 to 22, preferably 2 to 10, especially 3 to 4, carbon atoms. The hydroxy-functional compound of the formula R¹—OH constitutes the starter (the starter compound) for the alkoxylation reaction with the alkylene oxide(s), i.e. PO and optionally additionally EO. The alkylene oxides add onto the starter. The alkylene oxides are added onto the OH group in a polyaddition reaction with ring opening and particularly in the presence of a suitable catalyst. This leads to formation of the inventive compounds of the formula (I). The alkylene oxides may be added either individually in pure form, in alternating succession in any metering sequence, or else simultaneously in mixed form. This determines the sequence of the oxyalkylene units or alkyleneoxy units as repeat units in the polyether chain that forms. By the process, it is possible to construct polyether chains having the feature of controlled and reproducible preparability in terms of structure and molar mass. The sequence of repeat units can be varied by the sequence of addition of the alkylene oxides within broad limits. The molar mass of the compound of the formula (I) may be varied within wide limits and controlled in a controlled and reproducible manner via the molar ratio or mass ratio of the alkylene oxides in relation to the starter R¹—OH. The composition of compounds of the formula (I) can thus be adjusted via the stoichiometry. For example, the reaction product of 296 g (4 mol) of n-butanol, 870 g (15 mol) of propylene oxide and 660 g (15 mol) of ethylene oxide is a compound of the formula R¹O—[(C₂H₃R²)—O]ₙ—H with n=7.5 and R¹=n-butyl, where 50% of the R² radicals are methyl radicals. The HLB value of this compound is 7.2. The correlations between metered addition and product structure are known to those skilled in the art.

For the alkoxylation reaction, i.e. the reaction of the compound R¹—OH with PO and optionally additionally EO, it is possible in principle to use any of the alkoxylation catalysts known to the person skilled in the art, for example basic catalysts such as alkali metal hydroxides such as NaOH and KOH, alkali metal alkoxides such as sodium methoxide and potassium methoxide, amines, guanidines, amidines, phosphorus compounds such as triphenylphosphine, and additionally acidic and Lewis-acidic catalysts such as SnCl₄, SnCl₂, SnF₂, BF₃ and BF₃ complexes, and double metal cyanide (DMC) catalysts, especially those containing zinc hexacyanocobaltate(III). The DMC catalysts used are preferably the DMC catalysts described in U.S. Pat. No. 5,158,922, US 20030119663, WO 01/80994. The catalysts may be amorphous or crystalline.

After the alkoxylation reaction has ended, there particularly follows a period of further reaction for completion of the conversion. The further reaction can be conducted, for example, by continued reaction under reaction conditions (i.e. maintenance, for example, of the temperature and the pressure) without addition of reactants. particularly, the further reaction is effected with mixing of the reaction mixture, especially with stirring.

Unreacted alkylene oxides and any further volatile constituents can be removed after the alkoxylation reaction, for example, by vacuum distillation, steam or gas stripping, or other methods of deodorization.

The reaction product is particularly neutralized with an acid such as lactic acid, acetic acid, propionic acid or phosphoric acid, and the salts formed are optionally removed by filtration.

Reactor types used for the alkoxylation reaction may in principle be any suitable reactor types that allow control over the reaction and its exothermicity. The reaction regime may be continuous, semicontinuous or else batchwise in a manner known from chemical engineering and can be matched flexibly to the production equipment available. As well as conventional stirred tank reactors, it is also possible to use jet loop reactors with a gas phase and internal heat exchanger tubes as described in WO 01/062826. In addition, it is possible to use gas phase-free loop reactors.

The average molar mass (number-average molar mass Mn or mass-average molar mass Mw) and polydispersity Mw/Mn of the compounds of formula (I) is adjustable within wide limits via the molar ratio of the alkylene oxides to the starter R¹—OH used. It is preferable that the number-average molar mass of the at least one compound of the formula (I) is from 300 g/mol to 4500 g/mol, more particularly from 400 g/mol to 3000 g/mol, particularly from 800 g/mol to 2000 g/mol, especially from 1200 g/mol to 1500 g/mol, where the number-average molar mass is particularly determined as described in the examples. It is further preferable that the polydispersity Mw/Mn is from 1.0 to 3.0, particularly from 1.02 to 2.0, especially from 1.03 to 1.6, where the polydispersity is particularly determined as described in the examples. The compounds of formula (I) are liquid, pasty or solid according to the composition and molar mass.

The carrier may contain further constituents that differ from the at least one compound of the formula (I). For example, the carrier may contain defoamers, for example selected from the group of the water-insoluble hydrophobic compounds. Defoamers used may, for example, be silicone oils, organomodified siloxanes, mineral oils, vegetable oils and modified vegetable oils. In addition, further constituents present may be solids that affect the rheological properties, for example silica. Further constituents may be antioxidants. Biocides may additionally be present, provided that they do not impair the active ingredient. Further constituents present may additionally be water-absorbing substances, in order to even further improve storage and, later on in the application, dilutability with water if appropriate, to facilitate handling, to provide additional humectant properties and/or to prevent crystallization of active substances. Further constituents may be dispersion additives having what are called anchor groups for solids, for example sulfonates, phosphates, aromatic groups, hydroxyl groups. The dispersing additives should preferably not be surfactants. For the reasons discussed above, surfactants should preferably not be present in the carrier as further constituents other than the at least one compound of the formula (I). What are meant by surfactants in the context of the present disclosure are interface-active substances which, when mixed with water in a concentration of 0.5% by weight at a temperature of 20° C. and left to stand under those conditions for one hour,
a) give a transparent or translucent liquid or a stable emulsion without separation of insoluble substances and
b) lower the surface tension of water to 4.5×10⁻² N/m (45 dyn/cm) or less.

This corresponds to the definition of interface-active substances according to Customs Tariff Number Position 3402 (European Union). Surface tension can be ascertained, for example, with a tensiometer, which ascertains surface tension via the shape of a pendant drop (pendant drop method, drop contour analysis). An example of a suitable tensiometer is model OCA 25 (DataPhysics).

The Gram-negative bacteria according to any aspect of the present invention may be from a species especially beneficial for agricultural applications, like N-fixating bacteria. In particular, Gram-negative bacteria according to any aspect of the present invention may be selected from the group consisting of Agrobacterium sp., Azospirillum sp. Azotobacterium sp., Bacteroides sp., Bradyrhizobium sp., Burkholderia sp., Chromobacterium sp Curvibacter sp., Fusobacterium sp., Herbaspirillum sp., Janthinobacterium sp., Luteibacter sp., Lysobacter sp., Massilia sp., Mesorhizobium sp, Mitsuaria sp., Pseudomonas sp., Paenibacillus sp., Rhizobium sp., Salmonella sp., Serratia sp., Sinorhizobium sp., Sphingomonas sp., Stenotrophomonas sp. and Variovorax sp. More in particular, the Gram-negative bacteria may be selected from the group consisting of Acinetobacter baumani, Agrobacterium radiobacter, Agrobacterium tumefaciens, Azotobacter chroococcum, Azospirillum brasiliense, Bradyrhizobium arachidis, Bradyrhizobium japonicum, Bordetella pertussis, Brucella abortus, Brucella melitensis, Brucella suis, Burkholderia A39, Burkhulderia rinojensis, Campylobacter coli, Campylobacter foetus, Campylobacter jejuni, Chromobacterium subsugae, Curvibacter gracilis, Escherichia coli, Francisella tularensis, Haemophilus aphrophilus, Haemophilus haemolyticus, Haemophilus influenzae, Haemophilus parahaemolyticus, Haemophilus parainfluenza, Helicobacter pylori, Herbaspirillum hiltneri, Herbaspirillum lusitanum, Herbaspirillum rhizosphaerae, Klebsiella pneumoniae, Legionella pneumophilia, Luteibacter rhizovicina, Mesorihizobium cicero, Neisseria gonorrheae, Neisseria meningitidis, Pasteurella multocida, Pseudomonas aeruginosa, Pseudomonas azotoformans, Pseudomonas baetica, Pseudomonas brassicacearum, Pseudomonas brenneri, Pseudomonas cholororaphis, Pseudomonas fluorescens, Pseudomonas gessardii, Pseudomonas grimontii, Pseudomonas koreensis, Pseudomonas lini, Pseudomonas sp. JD18, Pseudomonas marginalis, Pseudomonas moraviensis, Pseudomonas palleroniana, Pseudomonas poae, Pseudomonas protegens, Pseudomonas psychrotolerans, Pseudomonas putida, Pseudomonas reinekei, Pseudomonas salomonii, Pseudomonas thivervalensis, Pseudomonas trivialis, Pseudomonas umsongensis, Pseudomonas viridiflava, Rhizobium azooxidifex, Rhizobium leguminosarum, Rhizobium lusitanum, Rickettsia rickettsii, Salmonella typhimurium, Serratia plymuthica, Shigella boydii, Shigella dysenteriae, Shigella flexneri, Shigella sonnei, Sinorhizobium meliloti, Sphingomonas PDD-69b-4, Sphingomonas strain A3K041, Stenotrophomonas rhizophila, Variovorax ginsengisoli, Variovorax paradoxus, Vibrio cholerae, Vibrio opticus, Yersinia enterocolitica, Proteus mirabilis, Yersinia pestis, and Yersinia pseudotuberculosis.

Even more in particular, the Gram-negative bacteria is selected from the group consisting of Agrobacterium tumefaciens, Azotobacter chroococcum, Azospirillum brasiliense, Bradyrhizobium japonicum, Curvibacter gracilis, Herbaspirillum hiltneri, Herbaspirillum lusitanum, Herbaspirillum rhizosphaerae, Luteibacter rhizovicina, Pseudomonas aeruginosa, Pseudomonas azotoformans, Pseudomonas baetica, Pseudomonas brassicacearum, Pseudomonas brenneri, Pseudomonas fluorescens, Pseudomonas gessardii, Pseudomonas grimontii, Pseudomonas koreensis, Pseudomonas lini, Pseudomonas sp. JD18, Pseudomonas marginalis, Pseudomonas moraviensis, Pseudomonas palleroniana, Pseudomonas poae, Pseudomonas protegens, Pseudomonas psychrotolerans, Pseudomonas reinekei, Pseudomonas salomonii, Pseudomonas thivervalensis, Pseudomonas trivialis, Pseudomonas umsongensis, Pseudomonas viridiflava, Rhizobium azooxidifex, Rhizobium leguminosarum, Rhizobium lusitanum, Serratia plymuthica, Stenotrophomonas rhizophila, Variovorax ginsengisoli, and Variovorax paradoxus.

By first spray-drying the cells in step (a) according to any aspect of the present invention and reducing the water content of the cells, before contacting the cells with the compound of Formula (I), the Gram-negative bacterial cells were found to have increased viability and thus also shelf life.

The compounds of the formula (I) may optionally contain small amounts of water (for example as a result of the synthesis or absorption of air humidity in the course of storage). It may therefore be advantageous to adjust, especially to reduce, the water content and hence the activity of water. This can be accomplished, for example, by means of a thermal separation process. Thermal separation processes are known by this term to those skilled in the art and include all processes based on the establishment of a thermodynamic phase equilibrium. Preferred thermal separation processes are selected from the group consisting of distillation, rectification, adsorption, crystallization, extraction, absorption, drying and freezing-out, particularly methods of distillation and rectification. For drying, it is also possible to use desiccants such as molecular sieves, for example zeolites.

The use of the carrier composition according to any aspect of the present invention leads to an improvement in handling and dosability of the Gram-negative bacteria since the carrier composition at 25° C. may have a viscosity of less than 1 Pa·s, and at 0° C. may have a viscosity of less than 10 Pa·s.

In particular, the carrier composition at 25° C. has a viscosity of less than 1 Pa·s, and at 0° C. has a viscosity of less than 10 Pa·s. The viscosity is determined as described in the examples.

The use of the carrier composition according to any aspect of the present invention also leads to an increase in storage stability of the active (biological or microbiological) ingredient.

The storage stability is determined as described in the examples in WO2020/234035.

According to another aspect of the present invention, there is provided a method for increasing the storage stability of at least one Gram-negative bacteria by
(a) subjecting a wet mass of Gram-negative bacteria to spray drying; and
(b) contacting the spray-dried Gram-negative bacteria from step (a) to a compound, to form the liquid composition of Gram-negative bacteria,
wherein the compound has a general formula (I) of,

R¹O—[(C₂H₃R²)—O]ₙ—H Formula (I)
where
R¹ is a monovalent aliphatic radical having 1 to 22, preferably 2 to 10, especially 3 to 4, carbon atoms;
R² is in each case independently a hydrogen radical or a methyl radical; and
n is a number from 1 to 300, preferably from 5 to 100, especially from 10 to 30,
with the proviso that at least one R² radical is a methyl radical.

According to a further aspect of the present invention, there is provided a liquid composition of Gram-negative bacteria comprising
(a) a spray-dried mass of Gram-negative bacteria with an activity of water of 0.05 to 0.4;
(b) a silica,
(c) a sugar; and
(d) a compound,
wherein the compound has a general formula (I) of,

R¹O—[(C₂H₃R²)—O]ₙ—H Formula (I)
where
R¹ is a monovalent aliphatic radical having 1 to 22, preferably 2 to 10, especially 3 to 4, carbon atoms;
R² is in each case independently a hydrogen radical or a methyl radical; and
n is a number from 1 to 300, preferably from 5 to 100, especially from 10 to 30,
with the proviso that at least one R² radical is a methyl radical.

One advantage is the improvement in the storability of Gram-negative bacteria through use of the carrier composition or improved storability of the liquid Gram-negative bacteria composition. More particularly, the liquid Gram-negative bacteria composition can be stored at room temperature for many weeks. This simplifies transport and storage. The composition is stored and transported, with exclusion of air, in bottles, pouches, canisters or drums that have been sealed airtight. The elevated storability especially leads to an increase in biological activity.

Furthermore, the liquid Gram-negative bacteria composition, especially in the form of a dispersion concentrate, suspension concentrate or oil dispersion, shows improved viability and/or germinability compared to the prior art.

A further improvement over the prior art is that the Gram-negative bacteria remain viable and/or germinable for much longer in the ready-to-use aqueous dilutions than in the aqueous dilutions based on the prior art.

A further advantage is that the carrier facilitates the dispersion of the Gram-negative bacteria in a liquid composition, for example the spray liquor.

A further advantage is that the carrier is self-emulsifying or water-soluble or can be rendered self-emulsifying or water-soluble. The carrier thus has good dilutability or can be rendered dilutable with water. The carrier composition and the active ingredient composition can be readily dispersed or even dissolved in water. Dilutability, i.e. self-emulsifiability or water solubility, is preferably determined as described in the examples. Self-emulsifying or water-soluble and hence dilutable carriers or active ingredient compositions can be dispersed or dissolved in water without any great input of shear. Self-emulsifying carriers here spontaneously form emulsion droplets, preferably with an average size of less than 400 μm, further preferably less than 200 μm, especially less than 100 μm. The size of the emulsion droplets can be determined, for example, by laser diffraction, for example by using laser diffraction systems or by computer-assisted image evaluation of high-resolution static images of the spray mist. The size of the emulsion droplets is preferably measured by laser diffraction, more preferably by using the MasterSizer 3000 from Malvern. The carrier can thus be efficiently dissolved or dispersed in water and forms a clear solution or a milky emulsion in water when the carrier and water are mixed in a mass ratio of 100:100 to 0.0001:100. Since efficiency enhancers for plant protection products are generally water-soluble in order thus to improve the efficacy of plant protection products from aqueous spray liquors, it is surprising in the light of the prior art that similar effects can also be achieved with self-emulsifying compositions. The self-emulsifying effect can especially be achieved by controlled adjustment of the hydrophobicity/hydrophilicity of the compound of the formula (I). In the case of tankmix formulations, there is sufficiently homogeneous distribution of the compound of the formula (I) in the spray liquor, for example even during the tankmixing operation. This facilitates firstly the preparation of spray liquors. Furthermore, it does not result in blockage of the spray nozzles as a result of the good incorporability and the associated homogeneous distribution during the spraying operation.

A further advantage is that the compound of the formula (I) has sufficiently low viscosity even at low temperature. This leads to better dosability and simpler handling of the plant protection product.

A further advantage is the biodegradability of many compounds of the formula (I), of the carrier and of the composition comprising the carrier and the Gram-negative bacteria. Biodegradability is preferably determined here by the OECD 301 F method. Further preferably, biodegradability is determined in accordance with OECD 301 F after 28 days at 22° C.

A further advantage is that the adhesion and retention of sprays/spray liquors containing the carrier composition or the liquid Gram-negative bacteria composition on plant surfaces that are difficult to wet is also improved.

A further advantage is the excellent compatibility of compounds of the formula (I) with conventional adjuvants and defoamers. Suitable adjuvants and defoamers are known to the person skilled in the art and are commercially available, for example, under the BREAK-THRU® (Evonik Industries AG), SURFYNOL® (Evonik Industries AG) and TOMADOL® (Evonik Industries AG) trade names.

Particularly good compatibility is found with BREAK-THRU® S 301 and BREAK-THRU® S 255.

Yet a further advantage is that the viscosity, rheology and/or sedimentation characteristics of the dissolved active ingredient can be adjusted in a simple manner by addition of silica. Suitable silicas are known to the person skilled in the art and are commercially available, for example, under the SIPERNAT® (Evonik Industries AG) trade name.

In particular, the sugar according to any aspect of the present invention may be an isomalt. The presence of the sugar and/or silica maintains the low aW of the final product, the liquid composition of the Gram-negative bacteria. Further, the presence of silica has the added benefit of being able to absorb water, has good handling properties/flowability making it suitable and easy to use. Further, silica increases the volume of the final product making the handling of the final product also easier and more convenient. Similarly, the presence of sugar in the final product forms a kind of covering or layer over the Gram-negative bacteria by glassification (possibly forming a quasi “glass coating”) to possibly reduce water influx and maybe also acting as outgrowth agent for the cells after the storage.

A varied concentration of silica and/or sugar may be present in the liquid composition of Gram-negative bacteria. WO2020104612A1 shows a range of concentrations of silica and/or sugar that may be applicable according to any aspect of the present invention. In one example, the percentage of silica may be about 6.1% (g/g) and the percentage of sugar may be about 15.91% (g/g).

It is preferable that the liquid composition of Gram-negative bacteria consists (essentially) of components (a) and (d).

In particular, the proportion by mass of the carrier, based on the total mass of the liquid composition of Gram-negative bacteria, is from 40% to <100%, particularly from 70% to <99.999%, more particularly from 80% to 99.99%.

More in particular, the proportion by mass of the Gram-negative bacteria, based on the total mass of the liquid composition of Gram-negative bacteria, is from >0% to 60%, particularly from 0.001% to 30%, more particularly from 0.01% to 20%.

The proportion by mass of the carrier based on the total mass of the liquid composition of Gram-negative bacteria is from 40% to <100%, particularly from 70% to <99.999%, more particularly from 80% to 99.99%, and that the proportion by mass of the Gram-negative bacteria, based on the total mass of the liquid composition of Gram-negative bacteria, is >0% to 60%, particularly from 0.001% to 30%, more particularly from 0.01% to 20%.

In particular, the proportion by mass of all compounds of the formula (I) based on the total mass of the liquid composition of Gram-negative bacteria is from 40% to <100%, particularly from 70% to <99.999%, more particularly from 80% to 99.99%, and/or the proportion by mass of all Gram-negative bacteria based on the total mass of the liquid composition of Gram-negative bacteria is from >0% to 60%, particularly from 0.001% to 30%, more particularly from 0.01% to 20%.

Particularly, the a_{w} value of the liquid composition of Gram-negative bacteria is less than 0.4, more particularly less than 0.3, even more particularly less than 0.25.

The liquid composition of Gram-negative bacteria is in liquid form, i.e. for example, in the form of an oil dispersion (OD), dispersion concentrate (DC) or suspension concentrate (SC). This has the advantage that the composition is easy to handle. But it is also possible that the liquid composition of Gram-negative bacteria is solid, i.e., for example, is in the form of a wettable powder (WP) or of water-dispersible granules (WG). In one example, the liquid composition of Gram-negative bacteria is in the form of an oil dispersion (OD).

The liquid composition of Gram-negative bacteria is obtainable by mixing the Gram-negative bacteria with the carrier. It is preferable that the gram-negative bacteria are dissolved and/or suspended and/or dispersed in the carrier.

The Gram-negative bacteria may be cultivated beforehand on a nutrient medium suitable for the purpose by methods known per se, for example submerged fermentation or solid fermentation. In one example, the Gram-negative bacteria may be processed by suitable separation, drying, grinding and/or dispersion methods. In one example, after the cultivation, the Gram-negative bacteria are separated from the culture substrate. In another example, the Gram-negative bacteria is dried together with the culture substrate using spray-drying methods. There may then be a subsequent separation/isolation of the Gram-negative bacteria by methods known per se, such as sieving, filtration, windsifting, decanting and/or centrifugation methods. After the drying, the Gram-negative bacteria are suspended and/or dispersed in the carrier. Preferably, the liquid Gram-negative bacteria composition is produced by mixing the Gram-negative bacteria into the carrier, particularly in a mixing tank using a stirrer. This affords a liquid Gram-negative bacteria composition, for example an oil dispersion (OD), suspension concentrate (SC) or dispersion concentrate (DC). By selection of suitable compounds of the formula (I) and/or use of appropriate viscosity regulators, it is possible to adjust the viscosity such that at least only a reduced separation, if any, of the Gram-negative bacteria that have been mixed into the liquid formulation, preferably an OD, SC or DC formulation, can be observed.

The liquid Gram-negative bacteria composition may be diluted with water in the spray tank to give a spray liquor for application to plants or on or in the soil. The proportion by mass of the water based on the total mass of the spray liquor is preferably 80% to 99.99%, preferably 90% to 99.9%, especially 95% to 99%. Alternatively, the proportion by mass may be higher or lower, according to the application rate of the Gram-negative bacteria. The spray liquor should particularly be sprayed at a maximum of 1000 litres, more particularly 50 litres to 600 litres, even more particularly at 100 litres to 400 litres, of water per hectare, which is guided by the application rate of the Gram-negative bacteria and by the type and number of plants.

The liquid Gram-negative bacteria composition may be used:
i) for the treatment of plants and/or
ii) for the treatment of soils and/or
iii) as biostimulant.

In particular, the liquid Gram-negative bacteria composition may be used as a formulation for spray liquors, where the proportion by mass of the carrier composition based on the total mass of the spray liquor is 0.001% to 1%.

### EXAMPLES

The foregoing describes preferred embodiments, which, as will be understood by those skilled in the art, may be subject to variations or modifications in design, construction or operation without departing from the scope of the claims. These variations, for instance, are intended to be covered by the scope of the claims.

### (e) Materials and Methods

Devices used are shown in Table 1.

TABLE 1
Lab equipment used for cultivation, spray drying
and analyzing biomass in the described experiments.

| Equipment | Provider |
|---|---|
| DASGIP 1L vessels | Eppendorf Vertrieb Deutschland |
| | GmbH, Germany |
| Infors shaker Multitron Pro | Infors GmbH, Germany |
| Revolver rotator D-6050 | neoLab Migge GmbH, Germany |
| Balance Kern EW 12000 | Kern & Sohn GmbH, Germany |
| Magnetic Stirrer | IKA GmbH & Co. KG, Germany |
| Minishaker M52 | IKA GmbH & Co. KG, Germany |
| Pipettes (Research/Reference) | Eppendorf Vertrieb Deutschland |
| | GmbH, Germany |
| Mini Spray Dryer B-290 | Buchi Labortechnik AG, Switzerland |
| Centrifuge Sigma 4K15 | Sigma Laborzentrifugen GmbH, |
| | Germany |
| Photometer Genesysy | Thermo Scienfic, Germany |
| 10S UV-VIS | |
| Labmaster-Aw Neo | Novasina AG, Switzerland |
| Moisture Analyzer, classic plus | Mettler Toledo |
| Climate cabinet | Binder GmbH, Germany |

Materials used for formulation experiments of freshly grown biomass are shown in Table 2.

TABLE 2
Polyethers and alternative compounds used to
formulate biomass for storage and agricultural application.

| Material | Supplier |
|---|---|
| Polyether BREAK-THRU ® | Evonik Operations GmbH |
| BP787 (BT-BP787) | |
| NaCl | Carl Roth GmbH + Co. KG |
| Glycerol | Alfa Aeser |
| PEG400 | Carl Roth GmbH + Co. KG |

Polyether BREAK-THRU® BP787 is a compound/polyether of Formula (I). In particular, BREAK-THRU® BP787 is formed by combining butanol and sodium methoxide as shown in Table 3a below.

TABLE 3a
Formation of BREAK-THRU ® BP787.

| | | | | | Proportion | | | | | |
|---|---|---|---|---|---|---|---|---|---|---|
| | | | | | of R² | | | | | |
| | | | | | radicals | | | OH | | |
| | | | | | with R² = | | | number | Mw | |
| Starter | | m(EO) | m(PO) | m(EO)/ | CH₃ in | Index | | in mg | in | Mw/ |
| alcohol | Base | in g | in g | m(PO) | % ^{[3]} | n ^{[4]} | HLB ^{[5]} | KOH/g | g/mol | Mn |
| 90 g n- | 7.2 g | 748 | 1033 | 42/58 | 51 | 29 | 8.0 | 40.3 | 1386 | 1.06 |
| butanol | sodium | | | | | | | | | |
| | methoxide | | | | | | | | | |

[3] proportion of R2 radicals with R2=CH3 in the compound of formula (I) reported in %=molar proportion of PO based on the total amount of PO and EQ=n(PO)/(n(PO)+n(EO)), with n(PO)=m(PO)/M(PO) and n(EO)=m(EO)/M(EO) and M(PO)=59.08 g/mol and M(EO)=44.05 g/mol
[4] index n according to formula (I)=average chain length=average number of repeat units
[5] calculated by HLB=20·(1-ml/m)=20·((m-ml)/m)=20·mh/m=20·m(EO)/[m(starter alcohol)+m(EO)+m(PO)]

### Example 1

### Cell Cultivation

To always generate reliable results using cells it is very important to have a well-controlled cultivation method yielding always the same cell quality. We use the DASGIP-parallel-fermentation-system and the same fermentation protocol and -program for every cell production process. Depending on the microorganism to be cultivated an appropriate medium had to be chosen.

The Media are shown in Table 4.

TABLE 4
Media used to cultivate different
microorganisms for formulation trials.

| Organism | Medium | Temperature |
|---|---|---|
| Pseudomonas fluorescens | M1Ps | 30° C. |
| Pseudomonas putida | M1Ps | 30° C. |
| Escherichia coli | M1Es | 30° C. |
| Azospirillum brasilense | M1Az | 32° C. |
| Bradyrhizobium japonicum | M1Br | 28° C. |

### (f) M1Ps

| | |
|---|---|
| (NH₄)₂SO₄ | 2.20 g/kg |
| NaCl | 0.02 g/kg |
| MgSO₄ × 7 H₂O | 4.00 g/kg |
| CaCl₂ × 2 H₂O | 0.10 g/kg |
| KH₂PO₄ | 6.00 g/kg |
| Glucose | 15.0 g/kg |
| Trace elements TE-Ps | 30 L/kg |

### (g) TE-Ps

| | |
|---|---|
| ZnSO₄ × 7 H₂O | 0.200 g/kg |
| MnSO₄ × H₂O | 0.090 g/kg |
| Na₃Citrate × 2 H₂O | 2.000 g/kg |
| CuSO₄ × 5 H₂O | 0.100 g/kg |
| NiSO₄ × 6 H₂O | 0.002 g/kg |
| Na₂MoO₄ × 2 H₂O | 0.003 g/kg |
| H₃BO₃ | 0.030 g/kg |
| FeSO₄ × 7 H₂O | 4.000 g/L |

Dissolve in water to reach 1 kg and filter-sterilize.

### (h) M1Es

| | |
|---|---|
| KH₂PO₄ | 12.07 g/kg |
| Na₃Citrate × 2 H₂O | 2.240 g/kg |
| (NH₄)₂SO₄ | 4.300 g/kg |
| MgSO₄ × 7 H₂O | 3.000 g/kg |
| Yeast extract | 1.500 g/kg |
| (NH₃) Fe(III) Citrate | 0.170 g/kg |
| Glucose | 15.00 g/kg |
| Trace elements TE-Es | 5.000 mL/kg |

### (i) TE-Es

| | |
|---|---|
| HCL 37% | 80 mL/L |
| MnCl₂ × 4 H₂O | 1.90 g/L |
| ZnSO₄ × 7 H₂O | 1.90 g/L |
| NaEDTA × 2 H₂O (Titriplex III) | 0.90 g(L |
| H₃BO₃ | 0.30 g/L |
| Na₂MoO₄ × 2 H₂O | 0.30 g/L |
| FeSO₄ × 7 H₂O | 17.8 g/L |
| CuCl₂ × 2 H₂O | 0.20/L |

### (j) M1Az

| | |
|---|---|
| KH₂PO₄ | 4.000 g/L |
| K₂HPO₄ | 4.580 g/L |
| Yeast extract | 5.000 g/L |
| MgSO₄ × 7 H₂O | 0.500 g/L |
| NaCl | 0.100 g/L |
| CaCl₂ × 2 H₂O | 0.015 g/L |
| Na₂MoO₄ × 2 H₂O | 0.002 g/L |
| FeCl₃ | 0.010 g/L |
| Fructose | 10.0 /L |

### (k) M1Br

| | |
|---|---|
| NaH₂PO₄ × 2 H₂O | 0.157 g/L |
| MgSO₄ × 7 H₂O | 0.180 g/L |
| Na₂SO₄ | 0.250 g/L |
| NH₄Cl | 0.320 g/L |
| CaCl₂ × 2 H₂O | 0.013 g/L |
| Yeast extract | 0.250 g/L |
| HEPES | 1.300 g/L |
| MOPS | 1.100 g/L |
| FeCl₃ | 0.004 g/L |
| Mannitol | 20.00 g/L |

All of the aforementioned organisms were cultured firstly from a plate/cryo stock in medium (25 ml in a 250 ml baffled shake flask) at 28-32° C. and 200 rpm for about 20 h as the first tier preculture. From this culture 50 ml broth in a 500 ml shake flask was inoculated in a way that this second tier preculture started at an OD₆₀₀ of 0.2. This shake flask was incubated at 28-32° C., 200 rpm for about 7 h to reach an OD₆₀₀ of ˜3-7. In order to inoculate the reactors with an optical density of 0.7, the OD₆₀₀ of the second preculture stage was measured and the amount of culture required to act as inoculum was calculated.

The starting volume in the reactor was 300 mL, which was tempered to the desired temperature with adjusted pH and dissolved oxygen (DO). The system was programmed to keep a DO of 30% by adjusting stirrer speed first and air flow second. Detailed controller parameters are shown in Table 5. The pH value was kept constant at the desired pH value by feeding 12.5% ammonia solution or 2.5 M H₂SO₄, controlled by the DASGIP Control software. Growth conditions for different microorganisms are shown in Table 4.

After inoculation cells were grown to an optical density of OD₆₀₀˜15-20. These values are reached in the batch phase without any substrate limitation applied to the cells in less than 20 h time.

After the cells grew to the desired optical density a sample of 25 mL was taken and stored for direct-liquid-formulation experiments. The residual majority of the cells was harvested by centrifugation: cells were transferred aseptically to centrifuge buckets, the buckets tared, and cells pelleted by centrifugation for 20 min at 12 000 g. Residual moistness was measured using the Mettler-Toledo “Moisture Analyzer, classic plus” for 2 h at 90° C. This measurement gave information about the cell dry weight corresponding to the cell wet weight of the cell pellet. These data are important for the correct composition of the spray draying solution.

TABLE 5
Controller parameters for cell cultivation in a 1 L DASGIP
reactor using 300 mL starting volume in a batch process.

| Dissolved oxygen | | | | | |
|---|---|---|---|---|---|
| (DO) regulator | | pH regulator | | T regulator | |
| Preset | 0% | Preset | 0 ml/h | P | 6K |
| P | 0.1 | P | 5 | Ti | 8520 s |
| Ti | 300 s | Ti | 200 s | | |
| min | 0% | min | 0 ml/h | | |
| max | 100% | max | 40 ml/h | | |
| | | | XO2 | | |
| N (Rotation) | from | to | (gas mixture) | from | to |
| growth and | 0% | 30% | growth and | 0% | 100% |
| biotrans- | 400 rpm | 1500 rpm | biotrans- | 21% | 21% |
| formation | | | formation | | |
| F (gas flow rate) | from | to | | | |
| growth and | 15% | 80% | | | |
| biotrans- | 6 sL/h | 72 sL/h | | | |
| formation | | | | | |

### Example 2

### Spray Drying

For spray drying a feed solution had to be prepared which contained the cells and several additives found to stabilize the cells during the dehydration process. Sipernat 50, a silica carrier material, risumalt and arabic gum (Gummi Arabicum) as glass former were also needed as Luria Broth medium as nutrient supplement for the cells. The detailed composition of the spray drying feed solution is shown in Table 6. A premix was prepared from NaCl solution, Gummi Arabicum, Risumalt and Sipernate by mixing the NaCl solution and Gummi Arabicum, stirring for 1 h, adding risumalt and stirring for one more hour, adding the Sipernat 50 and pasteurize the mixture tor 2 h at 80° C. This Premix can be stored a few weeks for later use in different spray drying experiments. After adding the cells and LB medium a sample was withdrawn for CFU analysis.

TABLE 6
Composition ot spray drying feed solution.

| | [g] |
|---|---|
| 0.9% NaCl-solution | 35.61 |
| Gummi Arabicum | 5.71 |
| Risumalt | 15.97 |
| Sipernat 50 | 6.71 |
| Luria Broth | 18.00 |
| Cells (wet weight) | 18.00 |

NaCl solution was mixed with Gummi Arabicum by stirring for 1 h at room temperature. Risumalt® was added afterwards and again mixed by stirring for 1 h at room temperature, Sipernat® was added last and this premix pasteurized for 2 h at 80° C. This premix was prepared as stock solution before starting the experiment. Cells and LB medium were added after cooling down the premix and stirred again for 1 h at room temperature.

For the dehydration process itself the Büchi “Mini Spray Dryer B-290” was used. After integration of the device, the system was sterilized at 180° C. for 1 h. Afterwards pre-run was carried out by spraying water into the drying chamber using the parameters shown in Table 7, all according to the manufacturers manual. During this pre-run the mist jet was focused into the drying chamber. After these preparations the spray drying solution containing the cells was fed to the system. This procedure lasted for about 15 min. Applied parameters are shown in Table 8. In the spray dryer the cells were dehydrated very quickly. They pass the hot zone in such a short period of time, that they were not harmed by the heat very much. The sugars mixed into the spray drying solution formed a glass surrounding the cells, shielded them from the air and stabilized their proteins and DNA.

TABLE 7
Spray draying pre-run using pure water
to focus the mist jet into the drying chamber.

| Parameters | |
|---|---|
| Aspirator [%] | 100 |
| Q-Flow [sL/h] | 60 |
| Pump rate [%] | 12-17 |
| Outlet-Temperature [° C.] | 53 |
| Inlet-Temperature [° C.] | 80 |

TABLE 8
Spray drying parameters for the cell containing solution.

| Parameters | |
|---|---|
| Aspirator [%] | 100 |
| Q-Flow [sL/h] | 60 |
| Outlet-Temperature [° C.] | 53 |
| Inlet-Temperature [° C.] | 80 |
| Pump Power [%] | 17 |
| Throughput | 100 g/15 min |
| Water flush | ja |
| Air flush | ja |

The dehydrated material was collected by the glass cyclone and fell into the collecting vessel. This cell dust was used for the formulation trials. First of all, water activity of the spray dried material was measured using the “Labmaster -Aw Neo” produced by Novasina AG, Switzerland. The water activity should be <0.3.

### Example 3

### Production and Characterization of Polyether BREAK-THRU® BP787 and Other Compounds of Formula I (EO: Ethylene Oxide or Ethyleneoxy, P: Propylene Oxide or Propyleneoxy)

A 3 Liter autoclave was initially charged with allyl alcohol or n-butanol as starter alcohol and sodium methoxide or potassium methoxide as base under nitrogen, and this initial charge was heated to 80-90° C. while stirring. The reactor was evacuated down to an internal pressure of 400 mbar in order to remove any volatile ingredients present by distillation. Propylene oxide (PO) and/or ethylene oxide (EO) were metered in continuously while cooling and stirring at 110120° C. and maximum internal reactor pressure 4.0 bar (absolute) for 6 h. Continued reaction at 110-120° C. for 60 minutes was followed by degassing. Volatile components such as residual propylene oxide or ethylene oxide were distilled off under reduced pressure. The polyether was neutralized with phosphoric acid. Water was removed by distillation at <30 mbar and 100° C. under reduced pressure. The low-viscosity and colourless polyether was cooled down to below 80° C. and discharged through a filter. The amounts of starter alcohol, sodium methoxide, ethylene oxide (EO) and/or propylene oxide (P0) used and the characterization of the polyethers obtained can be found in Table 3b.

TABLE 3b
Formation of BREAK-THRUR BP787 and other compounds under Formula (I)

| | | | | | | Proportion | | | | | |
|---|---|---|---|---|---|---|---|---|---|---|---|
| | | | | | | of R² | | | | | |
| | | | | | | radicals | | | OH | | |
| | | | | | | with R² = | | | number | Mw | |
| | Starter | | m(EO) | m(PO) | m(EO)/ | CH₃ in | Index | | in mg | in | Mw |
| EG | alcohol | Base | in g | in g | m(PO) | % ^{[3]} | n ^{[4]} | HLB ^{[5]} | KOH/g | g/mol | /Mn |
| 10 | 200 g | 8.7 g | 1679 | 719 | 70/30 | 25 | 19 | 12.9 | 61.3 | 914 | 1.08 |
| | n- | sodium | | | | | | | | | |
| | butanol | methoxide | | | | | | | | | |
| 11 | 111 g | 6.0 g | 883 | 640 | 58/42 | 35 | 21 | 10.8 | 55.8 | 1035 | 1.07 |
| | n- | sodium | | | | | | | | | |
| | butanol | methoxide | | | | | | | | | |
| 12 | 90 g n- | 7.2 g | 748 | 1033 | 42/58 | 51 | 29 | 8.0 | 40.3 | 1386 | 1.06 |
| | butanol | sodium | | | | | | | | | |
| | | methoxide | | | | | | | | | |
| 13 | 200 g | 5.5 g | 0 | 936 | 0/100 | 100 | 6 | 0.0 | 137 | 410 | 1.06 |
| | n- | sodium | | | | | | | | | |
| | butanol | methoxide | | | | | | | | | |
| 14 | 200 g | 6.5 g | 0 | 1840 | 0/100 | 100 | 12 | 0.0 | 79.5 | 705 | 1.07 |
| | n- | sodium | | | | | | | | | |
| | butanol | methoxide | | | | | | | | | |
| 15 | 80 g n- | 11.3 g | 0 | 2210 | 0/100 | 100 | 35 | 0.0 | 32.2 | 1835 | 1.10 |
| | butanol | sodium | | | | | | | | | |
| | | methoxide | | | | | | | | | |
| 16 | 145 g | 6.8 g | 1665 | 586 | 74/26 | 21 | 19 | 13.9 | 60.2 | 932 | 1.07 |
| | allyl | sodium | | | | | | | | | |
| | alcohol | methoxide | | | | | | | | | |
| 17 | 100 g | 7.8 g | 1026 | 1416 | 42/58 | 51 | 28 | 8.1 | 39.4 | 1420 | 1.06 |
| | allyl | sodium | | | | | | | | | |
| | alcohol | methoxide | | | | | | | | | |
| 18 | 58 g | 10.5 g | 1742 | 2405 | 42/58 | 51 | 81 | 8.3 | 14.4 | 4140 | 1.22 |
| | allyl | potassium | | | | | | | | | |
| | alcohol | methoxide | | | | | | | | | |

### Example 4

### Formulation Experiments—Dry Matter Formulation (DMF)

For the DMF trials spray dried material was mixed with the formulation agents as described below in the comparative example 4 in the ratios shown in Table 9.

TABLE 9
Composition of the dry-matter-formulation trials.

| | Ratio agent/ | Amount | Amount of |
|---|---|---|---|
| Agent | dry matter | of agent | dry matter |
| 0.9% NaCl | 90% | 9 mL | 1 g |
| BREAK-THRU ® BP787 | 90% | 9 mL | 1 g |
| (as disclosed in Example 1) | | | |
| Glycerin | 90% | 9 mL | 1 g |
| PEG400 | 90% | 9 mL | 1 g |

As described for the Direct Liquid Formulation (DLF) below, samples were taken directly after mixing the spray dried material with the formulation agents and after certain periods of storage time. These samples were subjected to CFU analysis together with the sample of the spray drying solution withdrawn before the dehydration process in the spray dryer.

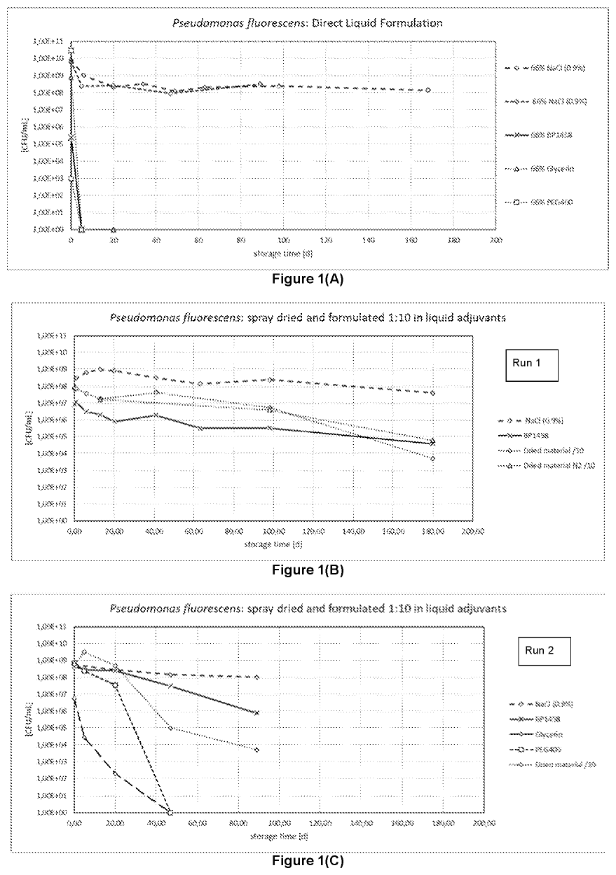

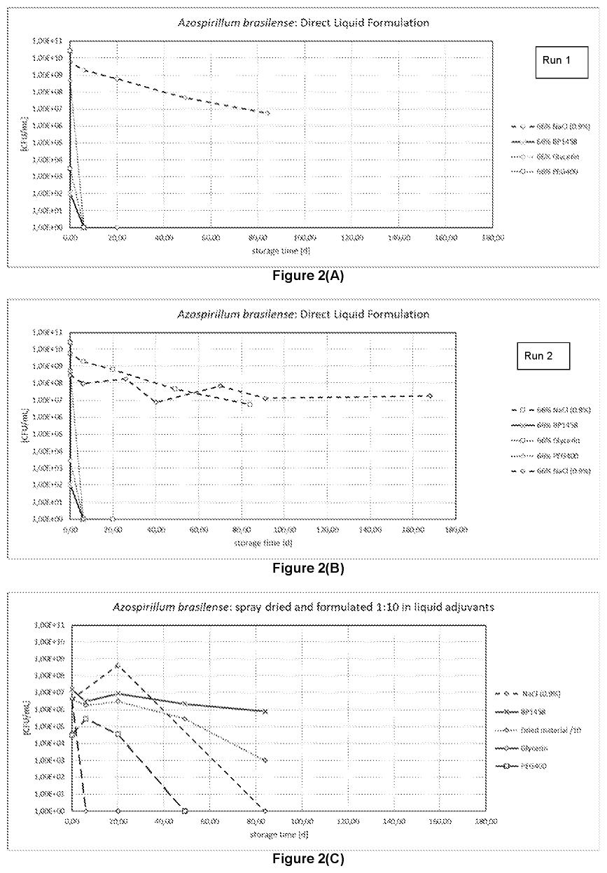

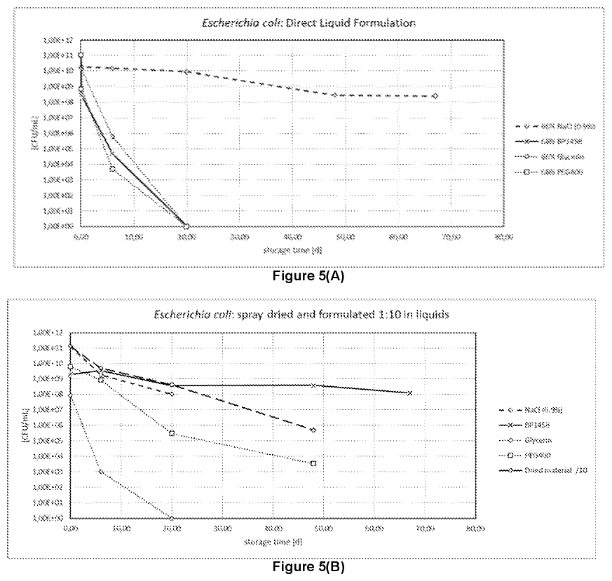

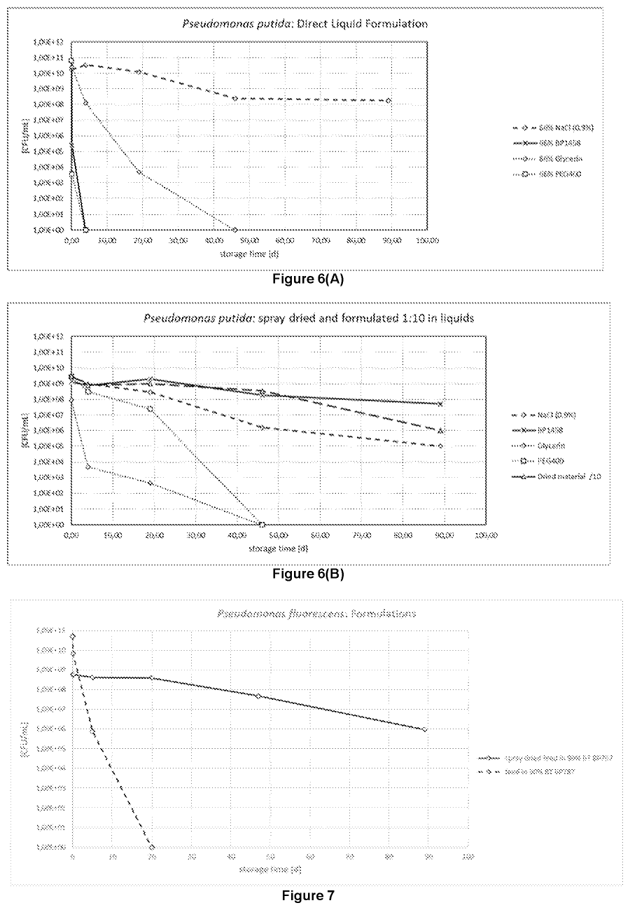

The results are shown in FIGS. 1B and C for Pseudomonas fluorescens, FIG. 2C for Azospirillum brasilense, FIG. 4B for Bradyrhizobium japonicum, FIG. 5B for Escherichia coli and FIG. 6B for Pseudomonas putida. After spray drying, the Aw of the spray dried material of Pseudomonas fluorescens, was 0.1589 and the survival rate after spray drying was 0.305% in Run 1, the Aw of the spray dried material of Pseudomonas fluorescens, was 0.2283 (residual moisture was 6.29%) and the survival rate after spray drying was 2.69% in Run 2. The residual moisture content was measured in every case in the examples with the Mettler Toledo Moisture Analyzer HB43-S (Germany) following the user manual. The weight of the sample was measured in the chamber at t=0. Then the sample was heated up by a halogen lamp, and the weight was continually measured, until a plateau was reached. The mass difference between t=0 and the plateau was the residual moisture content.

The A_{w} of the spray dried material of Azospirillum brasilense, was 0.1820 (residual moisture 6.04%) and the survival rate after spray drying was 0.02%. In contrast the A_{w} of spray dried material 1:10 in PEG was measured to be 0.2738 and after 52 days of storage, the A_{w} was 0.3903 as shown in FIG. 2C.

According to FIG. 4B, the A_{w} of the spray dried material of Bradyrhizobium japonicum, was 0.3442 and the survival rate after spray drying was 46.6%. As shown in FIG. 5B, the A_{w} of the spray dried material of Escherichia coli, was 0.2233 (residual moisture 7.8%). According to FIG. 6BB, the A_{w} of the spray dried material of Pseudomonas putida, was 0.1673 (residual moisture 5.58%) and the survival rate after spray drying was 10.35%.

As a control, all components used for spray drying (Table 6) were mixed, 90% BREAK-THRU® BP787 was added and subsequently stored at room temperature but without applying the spray drying process itself. The mixture reveals an A_{w} value of about 0.65 and the activity (CFU) of Pseudomonas fluorescens decreased rapidly upon storage at room temperature. Consequently, this is not sufficient to deliver high stability/survival rates to the cell. In contrast, when the same components (Table 6) are mixed, spray dried (survival rate: 2.69%) and subsequently mixed with 90% BREAK-THRU® BP787 the A_{w} of the product was found to be 0.2283 (residual moisture 6.29%) and stability of the spray dried material in 90% BREAK-THRU® BP787 is way higher than compared to the non-spray dried product.

Since dry material is not formulated in liquid adjuvant like described in the headline of the graph, the termₓ/10^{x} was added.

### Example 4 (Comparative Example)

### Formulation Experiments—Direct Liquid Formulation (DLF)

The sample of fermentation broth taken shortly before harvesting the cells was now used to prepare the first formulations: Broth was first centrifuged and the biomass pallet (33%) mixed with the different agents (66%) selected to be tested for their cell stabilizing properties as shown in Table 10. Mixing was achieved by vortexing for 10 sec. If this was not enough manual stirring using an inoculation loop was applied. Immediately after mixing a sample of each formulation was taken and used for CFU analysis to determine the start vitality of the cells.

TABLE 10
Composition of the direct-liquid-formulation trials.

| | Ratio | Amount | | Amount |
|---|---|---|---|---|
| Agent | agent/broth | of agent | | of broth |
| 0.9% NaCl | 66% | 6 | mL | 3 ml |
| BREAK-THRU ® BP787 | 66% | 6 | g | 3 ml |
| BREAK-THRU ® BP787 | 40% | 4 | g | 6 ml |
| Glycerin | 66% | 6 | g | 3 ml |
| PEG400 | 66% | 6 | g | 3 ml |

The formulations were than incubated in a “Revolver Rotater”, produced by neoLab Migge GmbH, Germany at 20° C. Further samples were taken after certain storage periods to observe the vitality pattern overtime. Those samples had to be taken aseptically always. They all were subjected to CFU analysis by plating dilutions prepared in LB medium and colony counting after incubation at 28-32° C. overnight.

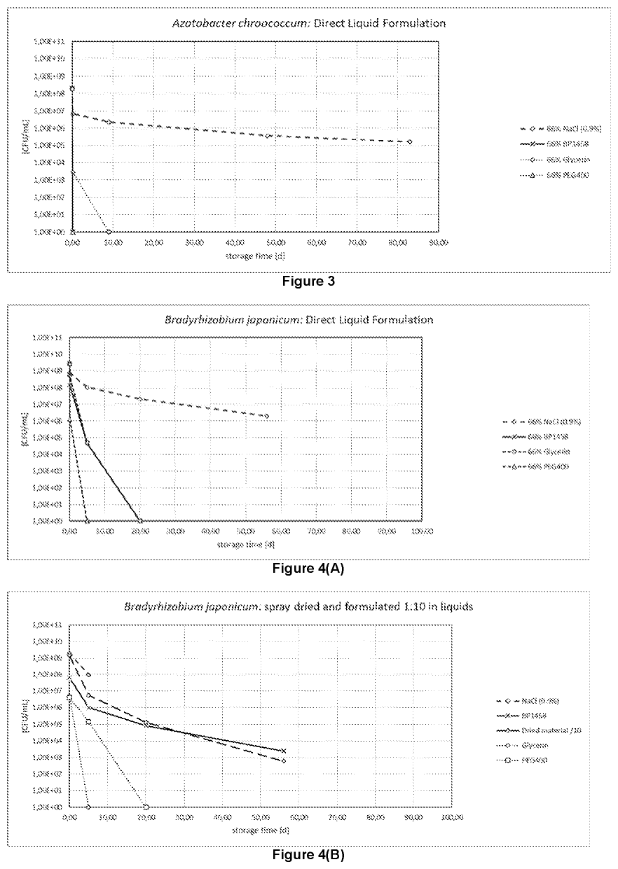

The results are shown in FIG. 1A for Pseudomonas fluorescens, FIGS. 2A and B for Azospirillum brasilense, FIG. 3 for Azotobacter chroococcum, FIG. 4A for Bradyrhizobium japonicum, FIG. 5A for Escherichia coli and FIG. 6A for Pseudomonas putida.

1. A method of producing a liquid composition of Gram-negative bacteria, the method comprising
(a) subjecting a wet mass of Gram-negative bacteria to spray drying; and
(b) contacting the spray-dried Gram-negative bacteria from step (a) to a compound,
wherein the compound has a general formula (I) of,
R¹O—[(C₂H₃R²)—O]ₙ—H Formula (I)
where
R¹ is a monovalent aliphatic radical having 1 to 22, preferably 2 to 10, especially 3 to 4, carbon atoms;
R² is in each case independently a hydrogen radical or a methyl radical; and
n is a number from 1 to 300, preferably from 5 to 100, especially from 10 to 30,
with the proviso that at least one R² radical is a methyl radical.

2. The method according to claim 1, wherein the R¹ radical is an alkyl radical.

3. The method according to claim 1, wherein the R¹ radical is selected from the group consisting of methyl, ethyl, n-propyl, iso-propyl, n-butyl, iso-butyl, sec-butyl, tert-butyl, n-pentyl (amyl), 2-pentyl (sec-pentyl), 3-pentyl; 2-methylbutyl, 3-methylbutyl (iso-pentyl or iso-amyl), 3-methylbut-2-yl, 2-methylbut-2-yl, 2,2-dimethylpropyl (neopentyl), hexyl, octyl, decyl, dodecyl, myristyl, stearyl, 2-ethylhexyl, 2-propylheptyl, 3,5,5-trimethylhexyl, isononyl, and isotridecyl.

4. The method according to claim 1, wherein 10% to 100% of the R² radicals are methyl radical.

5. The method according to claim 1, wherein the number-average molar mass of the at least one compound of the formula (I) is from 400 g/mol to 3000 g/mol.

6. The method according to claim 1, wherein the spray drying in step (a) is carried out in the presence of a silica, an isomalt and/or gum arabic.

7. The method according to claim 1, wherein the spray-dried Gram-negative bacteria after step (a) has a water activity of 0.05 to 0.4.

8. The method according to claim 1, wherein the spray-dried Gram-negative bacteria after step (a) has a water activity of about 0.4.

9. The method according to claim 1, wherein the Gram-negative bacteria is selected from the group consisting of Gram-negative bacteria selected from the group consisting of Agrobacterium sp., Azospirillum sp. Azotobacterium sp., Bacteroides sp., Bradyrhizobium sp., Burkholderia sp., Chromobacterium sp Curvibacter sp., Fusobacterium sp., Herbaspirillum sp., Janthinobacterium sp., Luteibacter sp., Lysobacter sp., Massilia sp., Mesorhizobium sp, Mitsuaria sp., Pseudomonas sp., Paenibacillus sp., Rhizobium sp., Salmonella sp., Serratia sp., Sinorhizobium sp., Sphingomonas sp., Stenotrophomonas sp. and Variovorax sp.

10. The method according to claim 1, wherein the Gram-negative bacteria is selected from the group consisting of Acinetobacter baumanii, Agrobacterium radiobacter, Agrobacterium tumefaciens, Azotobacter chroococcum, Azispirillum brasiliense, Bradyrhizobium arachidis, Bradyrhizobium japonicum, Bordetella pertussis, Brucella abortus, Brucella melitensis, Brucella suis, Burkholderia A39, Burkholderia rinojensis, Campylobacter coli, Campylobacter foetus, Campylobacter jejuni, Chromobacterium subsugae, Curvibacter gracilis, Escherichia coli, Francisella tularensis, Haemophilus aphrophilus, Haemophilus haemolyticus, Haemophilus influenzae, Haemophilus parahaemolyticus, Haemophilus parainfluenza, Helicobacter pylori, Herbaspirillum hiltneri, Herbaspirillum lusitanum, Herbaspirillum rhizosphaerae, Klebsiella pneumoniae, Legionella pneumophilia, Luteibactor rhizovicina, Mesorihizobium cicero, Neisseria gonorrheae, Neisseria meningitidis, Pasteurella multocida, Pseudomonas aeruginosa, Pseudomonas azotoformans, Pseudomonas baetica, Pseudomonas brassicacearum, Pseudomonas brenneri, Pseudomonas cholororaphis, Pseudomonas fluorescens, Pseudomonas gessardii, Pseudomonas grimontii, Pseudomonas koreensis, Pseudomonas lini, Pseudomonas sp. JD18, Pseudomonas marginalis, Pseudomonas moraviensis, Pseudomonas palleroniana, Pseudomonas poae, Pseudomonas protegens, Pseudomonas psychrotolerans, Pseudomonas putida, Pseudomonas reinekei, Pseudomonas salomonii, Pseudomonas thivervalensis, Pseudomonas trivialis, Pseudomonas umsongensis, Pseudomonas viridiflava, Rhizobium azooxidifex, Rhizobium leguminosarum, Rhizobium lusitanum, Rickettsia rickettsii, Salmonella typhimurium, Serratia plymuthica, Shigella boydii, Shigella dysenteriae, Shigella flexneri, Shigella sonnei, Sinorhizobium meliloti, Sphingomonas PDD-69b-4, Sphingomonas strain A3K041, Stenotrophomonas rhizophila, Variovorax ginsengisoli, Variovorax paradoxus, Vibrio cholerae, Vibrio opticus, Yersinia enterocolitica, Proteus mirabilis, Yersinia pestis, and Yersinia pseudotuberculosis.

11. A method for increasing the storage stability of at least one Gram-negative bacteria by
(a) subjecting a wet mass of Gram-negative bacteria to spray drying; and
(b) contacting the spray-dried Gram-negative bacteria from step (a) to a compound,
wherein the compound has a general formula (I) of,
R¹O—[(C₂H₃R²)—O]ₙ—H Formula (I)
where
R¹ is a monovalent aliphatic radical having 1 to 22, preferably 2 to 10, especially 3 to 4, carbon atoms;
R² is in each case independently a hydrogen radical or a methyl radical; and
n is a number from 1 to 300, preferably from 5 to 100, especially from 10 to 30,
with the proviso that at least one R² radical is a methyl radical.

12. The method according to claim 11, wherein the spray-dried Gram-negative bacteria of step (a) has a water activity of 0.05 to 0.4.

13. The method according to claim 11, of Gram-negative bacteria selected from the group consisting of Agrobacterium sp., Azospirillum sp. Azotobacterium sp., Bacteroides sp., Bradyrhizobium sp., Burkholderia sp., Chromobacterium sp Curvibacter sp., Fusobacterium sp., Herbaspirillum sp., Janthinobacterium sp., Luteibacter sp., Lysobacter sp., Massilia sp., Mesorhizobium sp, Mitsuaria sp., Pseudomonas sp., Paenibacillus sp., Rhizobium sp., Salmonella sp., Serratia sp., Sinorhizobium sp., Sphingomonas sp., Stenotrophomonas sp. and Variovorax sp.

14. The method according to claim 11, wherein the spray-drying in step (a) is carried out in the presence of a silica, an isomalt and/or gum arabic.

15. A liquid composition of Gram-negative bacteria comprising
(a) a spray-dried mass of Gram-negative bacteria with an activity of water of 0.05 to 0.4;
(b) a silica,
(c) an isomalt; and
(d) a compound,
wherein the compound has a general formula (I) of,
R¹O—[(C₂H₃R²)—O]ₙ—H Formula (I)
where
R¹ is a monovalent aliphatic radical having 1 to 22, preferably 2 to 10, especially 3 to 4, carbon atoms;
R² is in each case independently a hydrogen radical or a methyl radical; and
n is a number from 1 to 300, preferably from 5 to 100, especially from 10 to 30,
with the proviso that at least one R² radical is a methyl radical.
